(19) United States
(12) Patent Application Publication
(10) Pub. No.: US 2009/0004288 A1
(43) Pub. Date: Jan. 1, 2009

(54) STABILIZED OPHTHALMIC SOLUTIONS

(76) Inventors: Gary L. Collins, Jacksonville, FL (US); Shivkumar Mahadevan, Orange Park, FL (US); Frank F. Molock, JR., Orange Park, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/124,882

(22) Filed: May 21, 2008

Related U.S. Application Data
(60) Provisional application No. 60/947,184, filed on Jun. 29, 2007.

(51) Int. Cl. A61K 33/40

(52) U.S. Cl. 424/616

(57) ABSTRACT

The present invention relates to stabilized ophthalmic solutions comprising at least one peroxide. In one embodiment, the present invention relates to an ophthalmic solution comprising a pH between about 6 and about 8 and about 50 to about 1000 ppm hydrogen peroxide and about 0.005 wt % (50 ppm) to about 0.05 wt % (500 ppm) at least one salt of diethylenetriamine pentaacetic acid selected from the group consisting of calcium salts of diethylenetriamine pentaacetic acid, zinc salts of diethylenetriamine pentaacetic acid and mixed calcium/zinc salts of diethylenetriamine pentaacetic acid.

The present invention further relates to a method for preserving ophthalmic solution comprising at least one peroxide.

### REFERENCE TO RELATED APPLICATIONS

This application claims priority from U.S. provisional application Ser. No. 60/947,184, which is incorporated by reference in its entirety.

### BACKGROUND OF THE INVENTION

There are many commercially available ophthalmic solutions. The solutions must remain free from contamination during the life of the solution. To meet this requirement, solutions either contain a preservative component or are sterile packaged in single use dosages. For contact lenses cleaning and care solutions, and over the counter eye drops, multi-dose containers are popular. These solutions require the inclusion of preservatives (for eye drops) and disinfecting compositions (for contact lens cleaning and care solutions).

Hydrogen peroxide has been used as a disinfectant or preservative in ophthalmic solutions. However, hydrogen peroxide is not stable, and must either be included in concentrations which sting the eye or the solutions must contain additional components to stabilize the hydrogen peroxide. Compounds disclosed to be useful as peroxide stabilizers include phosphonates, phosphates, and stannates, and specific examples include physiologoically compatible salts of phosphonic acids such as diethylenetriamine pentamethylenephosphonic acid. Amino polycarboxylic acid chelating agents, such as ethylene diamine tetraacetic acid have also been disclosed.

Diethylenetriamine pentamethylenephosphonic acid (PTPPA) and ethylenediamine tetraacetic acid (EDTA) are cyctotoxic at relatively low levels and have low pH. Thus, these stabilizers can be included only in small amounts, and require the addition of neutralizing agents to provide a solution which is compatible with the human eye.

Accordingly, for solutions which are instilled directly in the eye, or for contact lens cleaning and care solutions which do not need to be rinsed off before the lens is placed on the eye, additional hydrogen peroxide stabilizers are desired.

### SUMMARY OF THE INVENTION

An ophthalmic composition comprising about 50 to about 1000 parts per million (ppm) hydrogen peroxide and about 0.005 wt % (50 ppm) to about 0.05 wt % (500 ppm) of at least one diethylenetriamine pentaacetic acid salt, selected from the group consisting of calcium salts of diethylenetriamine pentaacetic acid, zinc salts of diethylenetriamine pentaacetic acid, and mixed salts of diethylenetriamine pentaacetic acid comprising at least two salts selected from calcium, zinc and sodium.

An ophthalmic composition comprising a pH between about 6 and about 8 and about 50 to about 1000 parts per million (ppm) hydrogen peroxide and about 0.005 wt % (50 ppm) to about 0.05 wt % (500 ppm) of at least one diethylenetriamine pentaacetic acid salt, selected from the group consisting of calcium salts of diethylenetriamine pentaacetic acid, zinc salts of diethylenetriamine pentaacetic acid, and mixed salts of diethylenetriamine pentaacetic acid comprising at least two salts selected from calcium, zinc and sodium.

The present invention further relates to a method for preserving an ophthalmic composition comprising providing in said solution about 50 to about 1000 ppm hydrogen peroxide and about 0.005 wt % (50 ppm) to about 0.05 wt % (500 ppm) of at least one diethylenetriamine pentaacetic acid salt, selected from the group consisting of calcium salts of diethylenetriamine pentaacetic acid, zinc salts of diethylenetriamine pentaacetic acid and mixed calcium/zinc salts of diethylenetriamine pentaacetic acid.

### DESCRIPTION OF THE INVENTION

The present invention relates to a method for stabilizing ophthalmic solutions comprising low concentrations of hydrogen peroxide. The present invention further relates to ophthalmic solutions comprising small concentrations of hydrogen peroxide which are storage stable.

As used herein storage stable, means that under storage conditions, such as temperatures of less than about 40° C., the solution loses less than thirty percent over thirty days, and in some embodiments less than about 25% in thirty days.

Ophthalmic compositions are any compositions which can be directly instilled into an eye, or which can be used to soak, clean, rinse, store or treat any ophthalmic device which can be used or placed in or on the eye. Examples of ophthalmic compositions include ophthalmic device packing solutions, cleaning solutions, conditioning solutions, storage solutions, eye drops, eye washes, as well as ophthalmic suspensions, gels and ointments and the like. In one embodiment of the present invention, the ophthalmic composition is an ophthalmic solution.

Ophthalmic devices include any devices which can be placed on the eye, or any part of the eye, such as, but not limited to under the eyelid or in the puncta. Examples of ophthalmic devices include contact lenses, ophthalmic bandages, ophthalmic inserts, punctal plugs and the like.

The ophthalmic compositions of the present invention comprise between about 50 to about 1000 ppm hydrogen peroxide. In some embodiments the hydrogen peroxide is present in concentrations between about 100 and about 500 ppm, and in other embodiments, between about 100 and about 300 ppm.

Alternatively, the composition may include a source of hydrogen peroxide. Suitable hydrogen peroxide sources are known, and include peroxy compounds which are hydrolyzed in water.

It has been found that ophthalmic compositions comprising hydrogen peroxide in the amounts described above may be stabilized by including between about 0.005 wt % (50 ppm) to about 0.05 wt % (500 ppm) of at least one salt of diethylenetriamine pentaacetic acid comprising at least one calcium salt, zinc salt or mixed calcium/zinc salt of diethylenetriamine pentaacetic acid. As used herein, the term calcium salt, zinc salt or mixed calcium/zinc salt means that the DTPA comprises at least one of the specified cations. So for example, calcium salts of DTPA include DTPA salts which comprise at least one calcium ion. Examples include dicalcium salts of DTPA, dicalcium-trisodium salts of DTPA, and mixtures thereof. Examples of zinc salts of DTPA include, but are not limited to monozinc salts of DTPA. The salts of the present invention may further comprise any additional ophthalmically compatible cations such as sodium, magnesium, combinations thereof and the like. In one embodiment the DTPA salt comprises dicalcium DTPA. The concentration of the diethylenetriamine pentaacetic acid salt is between about 50 and about 300 ppm.

Surprisingly, dicalcium diethylenetriamine pentaacetic acid has been found to be at least as effective, and at some concentrations more effective at stabilizing hydrogen peroxide-containing ophthalmic solutions than diethylenetriamine pentamethylenephosphonic acid (DTPPA). Dicalcium diethylenetriamine pentaacetic acid is also less cytotoxic and has a more neutral pH than does DTPPA.

The ophthalmic compositions of the present invention also have a pH of between about 6 and 8, and in some embodiments between about 6.5 and about 7.5. This allows the compositions of the present invention to be instilled directly in the eye, and to be used on ophthalmic devices that are to be placed in the ocular environment.

The ophthalmic compositions may further comprise at least one additional peroxide stabilizer. Any known peroxide stabilizer may be used, so long as it is not cytotoxic at the concentrations being used, and is compatible with the other ophthalmic composition components. For example, the additional peroxide stabilizer should not interfere with the functioning of any other components included in the composition, and should not react with any other components. Examples of suitable additional peroxide stabilizers include phosphonates, phosphates, ethylene diamine tetraacetic acid, nitrilo triacetic acid, ophthalmically compatible water soluble salts of any of the foregoing, mixtures thereof, and the like. In one embodiment the additional peroxide stabilizer comprises DTPPA or least one pharmaceutically acceptable salt of DTPPA.

The at least one additional peroxide stabilizer may be present in concentrations up to about 1000 ppm, and in some embodiments between about 100 and about 500 ppm. When the additional peroxide stabilizer comprises DTPPA or at least one pharmaceutically acceptable salt of DTPPA, it is present in a concentration up to about 1000 ppm, and in some embodiments between about 100 ppm to about 500 ppm.

The ophthalmic compositions of the present invention may further comprise additional components such as, but not limited to pH adjusting agents, tonicity adjusting agents, buffering agents, active agents, lubricating agents, disinfecting agents, viscosity adjusting agents, surfactants and mixtures thereof. When the ophthalmic composition is an ophthalmic solution, all components in the ophthalmic solution of the present invention should be water soluble. As used herein, water soluble means that the components, either alone or in combination with other components, do not form precipitates or gel particles visible to the human eye at the concentrations selected and across the temperatures and pH regimes common for manufacturing, sterilizing and storing the ophthalmic solution.

The pH of the ophthalmic composition may be adjusted using acids and bases, such as mineral acids such as hydrochloric acid and bases such as sodium hydroxide.

The tonicity of the ophthalmic composition may be adjusted by including tonicity adjusting agents. In some embodiments it is desirable for the ophthalmic composition to be isotonic, or near isotonic with respect to normal, human tears. Suitable tonicity adjusting agents are known in the art and include alkali metal halides, phosphates, hydrogen phosphate and borates. Specific examples of tonicity adjusting agents include sodium chloride, potassium chloride, calcium chloride, magnesium chloride, zinc chloride, combinations thereof and the like.

The ophthalmic composition may further comprise at least one buffering agent which is compatible with diethylenetriamine pentaacetic acid salt. Examples of suitable buffering agents include borate buffers, sulfate buffers, combinations thereof and the like. In one embodiment the buffering agent comprises borate buffer.

The ophthalmic composition may also comprise at least one disinfecting agent in addition to hydrogen peroxide. The disinfecting agent should not cause stinging or damage to the eye at use concentrations and should be inert with respect to the other composition components. Suitable disinfecting components include polymeric biguanides, polymeric quarternary ammonium compounds, chlorites, bisbiguanides, quarternary ammonium compounds and mixtures thereof.

In one embodiment, the disinfecting component comprises at least one chlorite compound. Suitable chlorite compounds include water soluble alkali metal chlorites, water soluble alkaline metal chlorites and mixtures thereof. Specific examples of chlorite compounds include potassium chlorite, sodium chlorite, calcium chlorite, magnesium chlorite and mixtures thereof. In one embodiment the chlorite compound comprises sodium chlorite.

Suitable concentrations for the chlorite compound include concentrations between about 100 and about 2000 ppm, in some embodiments between about 100 and about 2000 ppm, between about 100 and about 1000 ppm, and in other embodiments between about 100 and about 500 ppm.

One or more lubricating agents may also be included in the ophthalmic composition. Lubricating agents include water soluble cellulosic compounds, hyaluronic acid, and hyaluronic acid derivatives, chitosan, water soluble organic polymers, including water soluble polyurethanes, polyethylene glycols, combinations thereof and the like. Specific examples of suitable lubricating agents include polyvinyl pyrrolidone, hydroxypropyl methyl cellulose, glycerol, polyethylene glycols, mixtures there of and the like. When a lubricating agent is used, it may be included in amounts up to about 5 weight %, and in some embodiments between about 100 ppm and about 2 weight %.

One or more active agents may also be incorporated into the ophthalmic solution. A wide variety of therapeutic agents may be used, so long as the selected active agent is inert in the presence of peroxides. Suitable therapeutic agents include those that treat or target any part of the ocular environment, including the anterior and posterior sections of the eye and include pharmaceutical agents, vitamins, nutraceuticals combinations thereof and the like. Suitable classes of active agents include antihistamines, antibiotics, glaucoma medication, carbonic anhydrase inhibitors, anti-viral agents, anti-inflammatory agents, non-steroid anti-inflammatory drugs, antifungal drugs, anesthetic agents, miotics, mydriatics, immunosuppressive agents, antiparasitic drugs, anti-protozoal drugs, combinations thereof and the like. When active agents are included, they are included in an amount sufficient to product the desired therapeutic result (a “therapeutically effective amount”).

The ophthalmic composition of the present invention may also include one or more surfactants. Suitable examples include poloxomer (poly(ethylene oxide)-b-poly(propylene oxide)-b-poly(ethylene oxide)) type surfactants which are commercially available from BASF and poloxamine type surfactants (non-ionic, tetrafunctional block copolymers based on ethylene oxide/propylene oxide, terminating in primary hydroxyl groups, commercially available from BASF, under the tradename Tetronic). A specific example is Pluronic F-147 and Tetronic 1304. Surfactants may be used in amounts up to about 5 weight %, and in some embodiments up to about 2 weight %.

Additionally, the ophthalmic composition may comprise one or more viscosity adjusting agent or thickener. Suitable viscosity adjusting agents are known in the art and include polyvinyl alcohol, polyethylene glycols, guar gum, combinations thereof and the like. The viscosity adjusting agent may be used in amounts necessary to achieve the desired viscosity.

Ophthalmic solutions of the present invention may be formed by mixing the selected components with water. Other ophthalmic compositions may be formed by mixing the selected components with a suitable carrier.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

### Examples 1-3 & Comparative Examples 1 and 2

The base solution shown in Table 1, below was made as follows. HPMC was weighed into about 100 ml deionized water and gently heated to allow all of the material to dissolve. The HPMC solution was allowed to cool and an additional ˜500 ml deionized water was added.

NaCl, boric acid, and poloxamer, were added to the solution in the amount listed in Table 1. Dequest 2060 (CAS 15827-60-8, from Fluka Sigma Aldrich) the dicalcium salt of DTPA (ISP Columbus) or a mixture of the two, were added in the amount listed in Table 2. The solution was mixed thoroughly until all components were fully dissolved. The solution was titrated with NaOH solution (0.1N) until the pH was 7.2-7.4.

Deionized water was added to make up a total of approximately 950 ml. The pH was checked and corrected to 7.2-7.4, if necessary. Sodium chlorite and hydrogen peroxide were added in the amounts listed in Table 1 and mixed thoroughly. The pH was rechecked and neutralized with NaOH solution as necessary. Deionized water was added to make up to 1000 g total. The solutions were stored in opaque polypropylene or high density polyethylene containers.

TABLE 1

| Component | Source | Weight (gm) |
|---|---|---|
| NaCl | Fisher Science ED | 7.5 |
| Boric Acid | Fisher Science ED | 1.5 |
| Poloxamer F-127 | BASF | 1 |
| Hydroxylpropyl | Acros Organics | 1.5 |
| methyl cellulose | | |
| (HPMC) | | |
| Sodium chlorite | Acros | 0.5 |
| Hydrogen peroxide | Fisher Scientific | 0.83 |
| (30%) | | |
| Purified water | | Q.S. 1000 |

100 g aliquots of the solution containing the amounts of DTPPA, DTPA or both, as shown in Table 2, below, were placed in opaque plastic containers and labeled.

A 5 ml sample from each container was removed and analyzed for hydrogen peroxide using the metavanadate calorimetric method, according to the method disclosed in Talanta, vol. 66, issue 1, pg 86-91, Mar. 31, 2005. This is the baseline (t=0) hydrogen peroxide concentration, reported in the fourth column of Table 2, below. Each container was weighed, and the baseline weights were recorded. The containers were stored at 40° C. At each of the intervals shown in Table 2, each container was weighed and 5 ml sample was removed for hydrogen peroxide determination as described above. The results are shown in Table 2. The value for Δppm was calculated by subtracting the concentration hydrogen peroxide in each solution measured at the time shown in Table 2, and subtracting from the original hydrogen peroxide concentration for that sample. The % Δ was calculated by dividing the concentration of hydrogen peroxide in each solution measured at the time shown in Table 2, by the original hydrogen peroxide concentration for that sample.

### Examples 4-9 and Comparative Examples 3-4

Examples 1-3 and Comparative Example 1 were repeated, except that 5 ppm of either iron sulfate or copper sulfate were added after the addition of stabilizer, but before the chlorite. Peroxide stability was evaluated as in Examples 1-3 and the results are shown in Tables 3 (copper) and 4 (iron), below.

TABLE 2

| | | | | Day 4 | | Day 9 | | Day 16 | | Day 29 | | Day 36 | |
|---|---|---|---|---|---|---|---|---|---|---|---|---|---|
| | | DTPA | Initial | [H₂0₂] | | [H₂0₂] | | [H₂0₂] | | [H₂0₂] | | [H₂0₂] | |
| | DTPPA | (mmol/ | [H₂0₂] | Δ | | Δ | | Δ | | Δ | | Δ | |
| Ex# | (mmol/100 ml) | 100 ml) | ppm | ppm | % Δ | ppm | % Δ | ppm | % Δ | ppm | % | ppm | % Δ |
| CE1 | 0 | 0 | 252 | −15 | 6 | −21 | 8 | −38 | 15 | −64 | 25 | −76 | 30 |
| CE2 | 0.02 | 0 | 243 | −15 | 6 | −22 | 9 | −39 | 16 | −64 | 26 | −71 | 29 |
| 2 | 0 | 0.02 | 257 | −17 | 7 | −15 | 6 | −31 | 12 | −55 | 21 | −64 | 25 |
| 3 | 0.01 | 0.01 | 258 | −16 | 6 | −17 | 7 | −35 | 14 | −59 | 23 | −67 | 26 |

TABLE 3
Copper addition (5 ppm)

| | | | | Day 4 | | Day 9 | | Day 16 | | Day 29 | | Day 36 | |
|---|---|---|---|---|---|---|---|---|---|---|---|---|---|
| | | DTPA | Initial | [H₂0₂] | | [H₂0₂] | | [H₂0₂] | | [H₂0₂] | | [H₂0₂] | |
| | DTPPA | (mmol/ | [H₂0₂] | Δ | | Δ | | Δ | | Δ | | Δ | |
| Ex# | (mmol/100 ml) | 100 ml) | ppm | ppm | % Δ | ppm | % Δ | ppm | % Δ | ppm | % | ppm | % Δ |
| CE3 | 0 | 0 | 179 | −179 | 100 | NA | 100 | NA | 100 | NA | 100 | NA | 100 |
| 4 | 0.02 | 0 | 243 | −22 | 9 | −24 | 10 | −42 | 17 | −66 | 27 | −74 | 30 |
| 5 | 0 | 0.02 | 250 | −12 | 5 | −14 | 6 | −28 | 11 | −50 | 20 | −60 | 24 |
| 6 | 0.01 | 0.01 | 239 | −15 | 6 | −14 | 6 | −30 | 13 | −54 | 23 | −61 | 26 |

TABLE 4
Iron addition (5 ppm)

| | | | | Day 4 | | Day 9 | | Day 16 | | Day 29 | | Day 36 | |
|---|---|---|---|---|---|---|---|---|---|---|---|---|---|
| | | DTPA | Initial | [H₂0₂] | | [H₂0₂] | | [H₂0₂] | | [H₂0₂] | | [H₂0₂] | |
| | DTPPA | (mmol/ | [H₂0₂] | Δ | | Δ | | Δ | | Δ | | Δ | |
| Ex# | (mmol/100 ml) | 100 ml) | ppm | ppm | % Δ | ppm | % Δ | ppm | % Δ | ppm | % | ppm | % Δ |
| CE4 | 0 | 0 | 250 | −34 | 14 | −35 | 14 | −54 | 22 | −80 | 32 | −90 | 36 |
| 7 | 0.02 | 0 | 244 | −16 | 7 | −18 | 7 | −35 | 14 | −55 | 23 | −63 | 26 |
| 8 | 0 | 0.02 | 251 | −15 | 6 | −17 | 7 | −36 | 14 | −62 | 25 | −71 | 28 |
| 9 | 0.01 | 0.01 | 254 | −16 | 6 | −14 | 6 | −31 | 12 | −56 | 22 | −63 | 25 |

The data in Table 2 above shows that peroxide solutions which are stabilized with the dicalcium salt of DTPA lose less peroxide than unstabilized solutions or solutions stabilized with DTPPA. The stabilized solutions of the present invention lose less than 25% and in some cases less than about 20% peroxide over about 30 days at 40° C. The data in Tables 3 and 4 show that peroxide solutions which are stabilized with the dicalcium salt of DTPA lose substantially less peroxide than unstabilized solutions. None of the solutions lost more than about 0.4 g due to evaporation during the course of the evaluation.

### Examples 10-11

Example 2 was repeated except that the concentration of the dicalcium salt of DTPA was varied as shown in Table 3, below, and the pH was not adjusted after the addition of the DTPA salt. At the intervals listed in Table 3, below, samples were withdrawn and tested as described for Example 2.

TABLE 3

| | | | | | Day 18 | | Day 29 | | Day 48 | |
|---|---|---|---|---|---|---|---|---|---|---|
| | | Initial | Day 7 | | [H₂0₂] | | [H₂0₂] | | [H₂0₂] | |
| Ex | | [H₂0₂] | Δ | | Δ | | Δ | | Δ | |
| # | DTPA (gm) | ppm | ppm | % Δ | ppm | % Δ | ppm | % Δ | ppm | % Δ |
| CE1 | 0 | 257 | −25 | 10 | −69 | 27 | −103 | 40 | −132 | 51 |
| 4 | 0.01 | 259 | −13 | 5 | −31 | 12 | −48 | 19 | −79 | 30 |
| 5 | 0.025 | 263 | −15 | 6 | −36 | 14 | −53 | 20 | −84 | 32 |

We claim:

1. An ophthalmic composition comprising a pH between about 6 and about 8 and about 50 to about 1000 ppm hydrogen peroxide and about 0.005 wt % (50 ppm) to about 0.05 wt % (500 ppm) of at least one diethylenetriamine pentaacetic acid salt, selected from the group consisting of calcium salts of diethylenetriamine pentaacetic acid, zinc salts of diethylenetriamine pentaacetic acid and mixed salts of diethylenetriamine pentaacetic acid comprising at least two salts selected from calcium, zinc and sodium.

2. The solution of claim 1 wherein said hydrogen peroxide is present in a concentration between about 100 and about 500 ppm.

3. The solution of claim 1 wherein said hydrogen peroxide is present in a concentration between about 100 and about 300 ppm.

4. The solution of claim 1 wherein said pH is between about 6.5 about 7.5.

5. The solution of claim 1 wherein said diethylenetriamine pentaacetic acid is present in a concentration between about 50 and about 300 ppm.

6. The solution of claim 1 further comprising water.

7. The solution of claim 1 further comprising diethylenetriamine pentamethylenephosphonic acid.

8. The solution of claim 7 wherein said diethylenetriamine pentamethylenephosphonic acid is present in a concentration up to about 1000 ppm.

9. The solution of claim 7 wherein said diethylenetriamine entamethylenephosphonic acid is present in a concentration between about 100 ppm to about 500 ppm.

10. The solution of claim 1 further comprising at least one additional stabilizer.

11. The solution of claim 1 wherein said at least one additional stabilizer is selected from phosphonates, phosphates, ethylene diamine tetraacetic acid, nitrilo triacetic acid, ophthalmically compatible water soluble salts of any of the foregoing and mixtures thereof.

12. The solution of claim 1 further comprising at least one additional component selected from the group consisting of tonicity adjusting agents, buffering agents, active agents, lubricating agents, disinfecting agents, surfactants and mixtures thereof.

13. The solution of claim 1 further comprising at least one buffering agent which is compatible with said dicalcium salt of diethylenetriamine pentaacetic acid.

14. The solution of claim 13 wherein said buffering agent is selected from the group consisting of borate buffers and sulfate buffers.

15. The solution of claim 13 wherein said buffering agent comprises borate buffer.

16. The solution of claim 1 further comprising at least one disinfecting agent selected from the group consisting of polymeric biguanides, polymeric quarternary ammonium compounds, chlorites, bisbiguanides, quarternary ammonium compounds and mixtures thereof.

17. The solution of claim 1 further comprising at least one chlorite compound.

18. The solution of claim 17 wherein said chlorite compound is selected from the group consisting of water soluble alkali metal chlorites, water soluble alkaline metal chlorites and mixtures thereof.

19. The solution of claim 17 wherein said chlorite compound is selected from the group consisting of potassium chlorite, sodium chlorite, calcium chlorite, magnesium chlorite and mixtures thereof.

20. The solution of claim 17 wherein said chlorite compound comprises sodium chlorite.

21. The solution of claim 17 wherein said chlorite compound is present in an amount between about 100 and about 2000 ppm.

22. The solution of claim 20 wherein said chlorite compound is present in an amount between about 100 and about 2000 ppm.

23. The solution of claim 20 wherein said chlorite compound is present in an amount between about 100 and about 1000 ppm.

24. The solution of claim 20 wherein said chlorite compound is present in an amount between about 100 and about 500 ppm.

25. The composition of claim 1 wherein said composition is an ophthalmic solution.

26. The composition of claim 1 wherein said salt of diethylenetriamine pentaacetic acid is selected from the group consisting of dicalcium salts of diethylenetriamine pentaacetic acid, dicalcium-trisodium salts of diethylenetriamine pentaacetic acid, monozinc salts of diethylenetriamine pentaacetic acid, and mixtures thereof.

27. The composition of claim 1 wherein said salt of diethylenetriamine pentaacetic acid is a dicalcium salt of diethylenetriamine pentaacetic acid.

28. A method for preserving an ophthalmic solution comprising providing in said solution about 50 to about 1000 ppm hydrogen peroxide and about 0.005 wt % (50 ppm) to about 0.05 wt % (500 ppm) at least one salt of diethylenetriamine pentaacetic acid selected from the group consisting of calcium salts of diethylenetriamine pentaacetic acid, zinc salts of diethylenetriamine pentaacetic acid and mixed salts of diethylenetriamine pentaacetic acid comprising at least two salts selected from calcium, zinc and sodium.

29. An ophthalmic composition comprising about 50 to about 1000 ppm hydrogen peroxide and about 0.005 wt % (50 ppm) to about 0.05 wt % (500 ppm) of at least one diethylenetriamine pentaacetic acid salt, selected from the group consisting of calcium salts of diethylenetriamine pentaacetic acid, zinc salts of diethylenetriamine pentaacetic acid and mixed salts of diethylenetriamine pentaacetic acid comprising at least two salts selected from calcium, zinc and sodium.
